(19) United States
(12) Patent Application Publication
(10) Pub. No.: US 2026/0256666 A1
(43) Pub. Date: Sep. 3, 2026

(54) Hair re-forming composition comprising malic acid and a betaine

(71) Applicant: WELLA GERMANY GMBH, Darmstadt (DE)

(72) Inventors: David HERMANN, Darmstadt (DE); Sonja TELSER, Darmstadt (DE); Katrin KAESTLE, Darmstadt (DE)

(21) Appl. No.: 19/157,852

(22) PCT Filed: Oct. 29, 2024
(86) PCT No.: PCT/EP2024/080587

§ 371 Date: Aug. 19, 2025

(30) Foreign Application Priority Data
Nov. 6, 2023 (EP) 23207954.1

(51) Int. Cl. A61K 8/365; A61K 8/19; A61K 8/41; A61K 8/44; A61K 8/46; A61Q 5/04

(52) U.S. Cl. CPC A61K 8/365; A61K 8/19; A61K 8/41; A61K 8/44; A61K 8/46; A61Q 5/04

(57) ABSTRACT

The presently claimed invention relates to a composition (CR) for permanently re-forming keratin fibers comprising as components (A) at least one thiol based reducing agent, (B) at least one alkalizing agent, (C) malic acid and/or salts of malic acid, (D) at least one betaine, and optionally (E) at least one disulfide based compound. Moreover, the presently claimed invention relates to a kit for permanently re-forming keratin fibers, comprising the composition (CR) for permanently re-forming keratin fibers and a neutralizing composition (CN) comprising at least one oxidizing agent. Another aspect of the presently claimed invention is a process for permanently re-forming keratin fibers.

The presently claimed invention relates to a composition (CR) for permanently re-forming keratin fibers comprising as components (A) at least one thiol based reducing agent, (B) at least one alkalizing agent, (C) malic acid and/or salts of malic acid, (D) at least one betaine, and optionally (E) at least one disulfide based compound. Moreover, the presently claimed invention relates to a kit for permanently re-forming keratin fibers, comprising the composition (CR) for permanently re-forming keratin fibers and a neutralizing composition (CN) comprising at least one oxidizing agent. Another aspect of the presently claimed invention is a process for permanently re-forming keratin fibers.

Keratin fibers, like human hair, differ in their morphological structure from hair roots to hair tips. This is because, among other things, of the effect of the environment like the action of the sun or weathering factors which may damage the keratin fibers. Other factors, that can damage keratin fibers are for example chemical and mechanical treatments of the hair like combing, brushing, washing or dye treatments. These external influences and the morphological changes of the keratin fibers cause an increased static charge of the hair, a poorer combability, a rough and unpleasant feel, a reduced gloss, and an increased brittleness of the hair. In extreme cases this can lead to breakage of the hair during or after the hair treatment.

Another well-known process for hair treatment are hair re-forming processes like permanent waving of keratin fibers or permanently straightening of keratin fibers.

The well-known and commonly used process for permanently re-forming keratin fibers, like hair, generally involves a reducing step in which a composition, comprising at least one reducing agent, is applied to the hair, and a subsequent oxidizing step, in which a composition, comprising at least one oxidizing agent is applied to the hair. Especially, the reducing step is often perceived as keratin fiber damaging.

There is a need for providing a composition for permanently re-forming keratin fibers, like hair, having a protective effect, especially during the reducing step.

The composition for permanently re-forming keratin fibers should preferably reduce the degree of hair degradation during the reducing step.

The object is solved by a composition (CR) for permanently re-forming keratin fibers comprising as components
(A) at least one thiol based reducing agent,
(B) at least one alkalizing agent,
(C) malic acid, and/or salts of malic acid,
(D) at least one betaine, and
(E) optionally at least one disulfide based compound.

Accordingly, another aspect of the presently claimed invention is drawn to a kit for permanently re-forming keratin fibers, comprising the composition (CR) for permanently re-forming keratin fibers as described herein, and a neutralizing composition (CN) comprising at least one oxidizing agent.

Another aspect of the presently claimed invention is drawn to a process for permanently re-forming keratin fibers, comprising the steps
i) applying the composition (CR) for permanently re-forming keratin fibers as described herein to keratin fibers,
ii) re-forming the keratin fibers, and
iii) applying the neutralizing composition (CN) comprising at least one oxidizing agent to the keratin fibers.

In one embodiment the composition (CR) for permanently re-forming keratin fibers may optionally comprise one, two or more of the following components:
(F) at least one cosmetically acceptable solvent,
(G) at least one surfactant, and
(H) at least one additional component.

### Definitions

As used in the specification and the appended claims, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term “and/or” in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers. According to embodiments, the term “about” is understood to mean±2 percent of the recited number, numbers or range of numbers.

As used herein, the term “optionally” means that the corresponding step or feature may or may not be present. It includes both possibilities.

The terms “parts-by-weight” (PBW) and “parts-by-volume” (PBV) are used in their usual meaning. When used in a mixed context, for example an amount of a component indicated in PBW in admixture with a component indicated in PBV, the terms PBW and PBV as used herein denote the amount in grams of the component indicated in PBW relative to a volume in milliliters of the amount of the component indicated in PBV.

The term “composition” as used herein denotes a mixture or blend comprising at least two components. Typical compositions comprise at least one functional component (such as, for example, an active ingredient such as malic acid and/or malic acid salts) and at least one solvent component (such as water or an alcohol). A composition may be present in any suitable physical form, including for example liquids of varying viscosities, gels, foams, creams, and the like. The term “composition” may denote a ready-to-use product unless the context dictates otherwise.

“Ready-to-use scalp-and-hair treatment product” denotes the form in which the composition is actually applied to human scalp, or a skin region comprising facial hair.

The term “essentially free” of a particular component denotes an amount of less than 1.0% by weight of the respective component in an entity such as a composition, based on the total weight of the entity. In particular, the term “essentially free” denotes an amount of less than 0.5% by weight, for example less than 0.1% by weight, such as less than 0.01% by weight of the respective component in the entity.

The term “free” of a particular component denotes an amount below the detection limit, assuming that detectability is on a ppm (parts per million) basis (by weight).

In particular, the term “free” of a particular component denotes an amount below 50 ppm by weight, for example less than 10 ppm by weight, such as less than 5 ppm by weight of the respective component in the entity.

The term “consisting essentially of” denotes the absence of additional, not specifically recited components in an entity such as a composition in an amount of more than 1.0% by weight, based on the total weight of the entity. In particular, the term “consisting essentially of” denotes the absence of additional, not specifically recited components in an amount of more than 0.5% by weight, for example more than 0.1% by weight, such as more than 0.01% by weight in an entity. It goes without saying that any additional, not specifically recited component is compatible with the specifically recited components and does not interfere with the function thereof.

The term “consisting of” denotes the absence of additional, not specifically recited components in an entity such as a composition in an amount below the detection limit, assuming that detectability is on a ppm (parts per million) basis (by weight).

In particular, the term “consisting of” denotes the absence of additional, not specifically recited components in an amount of more than 50 ppm by weight, for example more than 10 ppm by weight, such as more than 10 ppm by weight in an entity. It goes without saying that any additional, not specifically recited component is compatible with the specifically recited components and does not interfere with the function thereof.

The term “keratin fibers” as used herein denotes generally mammalian hair, but in particular human hair. More specifically, for the purposes of the present invention the term “hair” denotes human “head hair” or “scalp hair”, and human “facial hair”. The term “facial hair” encompasses beard hair, eyebrows and eyelashes.

The term “composition (CR) for permanently re-forming keratin fibers” and the term “composition (CR)” herein are used synonymously and, therefore, have the same meaning.

### DETAILED DESCRIPTION OF THE INVENTION

The inventors of the present invention surprisingly found that a combination of malic acid and/or malic acid salt(s) (component (C)), and at least one betaine (component (D)) is effective for protecting keratin fibers in a process for permanently re-forming keratin fibers. The inventors of the present invention surprisingly found that malic acid and/or malic acid salt(s) (component (C)), and at least one betaine (component (D)) are effective in a synergistic manner for protecting keratin fibers in a process for permanently re-forming keratin fibers.

### Component (A)

In the present case the terms “(A)”, “component (A)” and “at least one thiol based reducing agent” are used synonymously, and therefore have preferably the same meaning. The term “at least one thiol based reducing agent” means exactly one thiol based reducing agent and also mixtures of two or more different thiol based reducing agents.

The thiol based reducing agent (component (A)) is capable to reduce the disulfide cysteine bonds in keratin fibers especially in the cortex of the hair shaft, which is located between the hair cuticle and medulla and is the thickest hair layer. In a sense, the thiol based reducing agent (component (A)) removes cross links.

In one embodiment the thiol based reducing agent (component A)) is selected from the group consisting of thioglycolic acid, ammonium thioglycolate, thiolactic acid, glycerol monothiolactate, ammonium thiolactate, MEA-thiolactate, 3-mercaptopropionic acid, glycerol 3-mercaptopropionate, ethyleneglycol 3-mercaptopropionate, cysteamine, N-acetylcysteamine, N-propionylcysteamine, cysteine, N-acetylcysteine, N-alkanoylcysteine, cysteine alkyl esters, homocysteine, thioglycolic acid, ethanolamine thioglycolate, glyceryl thioglycolate, glutathione, thioglycerol, thiomalic acid, thiodiglycol, 2-mercaptoethanol, dithiothreitol, thioxanthine, thiosalicylic acid, thiopropionic acid, lipoic acid, and mixtures thereof.

In another preferred embodiment, the thiol based reducing agent (component (A)) is selected from the group consisting of thioglycolic acid, ammonium thioglycolate, cysteine, and mixtures thereof. Especially preferred thiol based reducing agents that may be used as component (A) are ammonium thioglycolate, and cysteine, wherein ammonium thioglycolate is especially preferred.

Ammonium thioglycolate is the ammonium salt of thioglycolic acid and has the CAS number 5421-46-5.

The amount of component (A) in the composition (CR) for permanently re-forming keratin fibers, preferably human hair, used according to the present disclosure is not particularly critical. Typically, component (A) will be present in an amount of from 1 to 15% by weight, based on the total weight of the composition (CR).

If the amount of component (A) is below 4% by weight, the effect sought by the present disclosure may be difficult to achieve. Amounts above 15% by weight may not further enhance the effect or may damage the keratin fibers, especially human hair, but contribute to higher costs of the material resources for the composition (CR).

In one embodiment component (A) is present in an amount in the range of 1 to 15 wt. %, preferably in an amount in the range of 1.5 to 14 wt. %, particular preferred in an amount in the range of 3 to 13 wt. %, and most preferred in an amount in the range of 5 to 13.5 wt. %, in each case based on the total weight of the composition (CR).

### Component (B)

In the present case “(B)”, “component (B)”, and “at least one alkalizing agent”, are used synonymously, and therefore, have preferably the same meaning. The term “at least one alkalizing agent” in the present case means exactly one alkalizing agent and also mixtures of two or more different alkalizing agents.

In a preferred embodiment in composition (CR) component (B) is at least one alkalizing agent selected form the group consisting of 2-amino-1-propanol, 2-amino-1-butanol, 2-dimethylamino-2-pethyl-1-propanol, 2-amino-2-methylpropan-1-ol, 3-amino-1-propanol, ammonia, and monoethanolamine, wherein ammonia, 2-amino-1-propanol and monoethanolamine are especially preferred.

In another preferred embodiment component (B) is ammonia or monoethanolamine, wherein ammonia is especially preferred.

In one embodiment component (B) is present in an amount in the range of 0.1 to 5 wt. %, preferably in an amount in the range of 0.15 to 4 wt. %, particular preferred in an amount in the range of 0.2 to 3 wt. %, and most preferred in an amount in the range of 0.25 to 2 wt. %, in each case based on the total weight of the composition (CR).

### Component (C)

In the present case the terms “(C)”, “component (C)”, and “malic acid, and/or salts of malic acid” are used synonymously, and therefore, have preferably the same meaning.

Malic acid is a dicarboxylic acid with the molecular formula C₄H₆O₅. Malic acid has two stereo enantiomeric forms, namely the L- and the D-enantiomer. Only the L-enantiomer exists naturally. Racemic malic acid can be produced by double hydration of maleic anhydride. The racemic malic acid comprises a mixture of the L- and the D-isomer of malic acid.

Malic acid and/or malic acid salt(s) is/are one of the active ingredients for protecting keratin fibers in a process for permanently re-forming keratin fibers.

As component (C) preferably racemic malic acid and/or salts of racemic malic acid is/are used. Suitable salts of malic acid, preferably racemic malic acid, are sodium or ammonium salts of malic acid, preferably sodium or ammonium salts of racemic malic acid.

In an especially preferred embodiment as component (C) racemic malic acid is used. The CAS number of racemic malic acid is 6915-15-7.

In one embodiment component (C) is present in an amount in the range of 0.1 to 15 wt. %, preferably in an amount in the range of 0.2 to 10 wt. %, particular preferred in an amount in the range of 0.25 to 8 wt. %, and most preferred in an amount in the range of 0.3 to 3 wt. %, in each case based on the total weight of the composition (CR).

### Component (D)

In the present case the terms “(D)”, “component (D)”, and “at least one betaine” are used synonymously, and therefore, have preferably the same meaning. The term “at least one betaine” in the present case means exactly one betaine and also mixtures of two or more different betaines.

The at least one betaine is another active ingredient for protecting keratin fibers in a process for permanently re-forming keratin fibers.

In one embodiment of the present invention the betaine (component (D)) may be selected from the group consisting of alkyl betaines, alkyl amidopropyl betaines, alkyl sulfo betaines, and mixtures thereof.

In a preferred embodiment the composition (CR) for permanently re-forming keratin fibers comprises as component (D) at least one betaine of the formula (I)

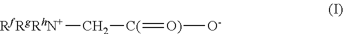

wherein R^{f}, R^{g} and R^{h} are each, same or different, selected from the group consisting of C1-C6 alkyl, C1-C₆ hydroxyalkyl and C1-C₆ carboxyalkyl.

In a preferred embodiment R^{f}, R^{g} and R^{h} in formula (I) are each, same or different selected from the group consisting of C1 to C6-alkyl, more preferred selected from the group consisting of C1 to C4-alkyl and especially preferred selected from the group consisting of C1 to C2-alkyl.

In an especially preferred embodiment component (D) is trimethylglycine. Trimethylglycine is an amino acid derivative. It was the first betaine discovered.

The IUPAC named of trimethylglycine is (trimethylammonio)acetate. The CAS number of trimethylglycine is 107-43-7.

In one embodiment component (D) is present in an amount in the range of 0.1 to 15 wt. %, preferably in an amount in the range of 0.2 to 10 wt. %, particular preferred in an amount in the range of 0.25 to 8 wt. %, and most preferred in an amount in the range of 0.3 to 3 wt. %, in each case based on the total weight of the composition (CR).

### Component (E)

In the present case the terms “(E)”, “component (E)”, and “at least one disulfide based compound” are used synonymously, and therefore, have preferably the same meaning. The term “at least one disulfide based compound” in the present case means exactly one disulfide based compound and also mixtures of two or more different disulfide based compounds. In a preferred embodiment, if component (E) is present in the composition (CR) exactly on disulfide compound is used.

In one embodiment of the present invention the disulfide based compound (component (E)) may be selected from the group consisting of dithiodiglycolic acid, derivatives thereof, and mixtures thereof.

Non-limiting examples include dithiodiglycolic acid, diammonium dithiodiglycolate, and mixtures thereof. In a preferred embodiment component (E), if present in the composition (CR), is diammonium dithiodiglycolate.

In one embodiment component (E) is present in an amount in the range of 0 to 10 wt. %, preferably in an amount in the range of 0.1 to 8 wt. %, particular preferred in an amount in the range of 0.5 to 6 wt. %, and most preferred in an amount in the range of 1 to 4 wt. %, in each case based on the total weight of the composition (CR).

### Component (F)

In the present case the terms “(F)”, “component (F)” and “at least one cosmetically acceptable solvent” are used synonymously, and therefore have preferably the same meaning. The term “at least one cosmetically acceptable solvent” means exactly one cosmetically acceptable solvent and, also a mixture of two or more different cosmetically acceptable solvents. Preferably as component (F) a mixture of two or more different cosmetically acceptable solvents is used.

The cosmetically acceptable solvent used in the composition (CR), preferably is water-based, alcohol-based or water-alcohol-based. Typically, the cosmetically acceptable solvent comprises water and one or more monovalent and/or polyvalent C1-C4 alcohols. According to embodiments, the solvent comprises water and an alcohol selected from ethanol, n-propanol, isopropanol, glycerol, ethylene glycol, propylene glycol, butylene glycol, ethoxdiglycol or a combination thereof.

In one embodiment component (F) is present in an amount in the range of 30 to 98.7 wt. %, preferably in an amount in the range of 45 to 96.949 wt. %, particular preferred in an amount in the range of 54 to 94.29 wt. % and most preferred in an amount in the range of 65.5 to 91.55 wt. %, in each case based on the total weight of the composition (CR).

In case the composition (CR) comprises at least one disulfide based component (component (E)) in one embodiment component (F) is present in an amount in the range of 20 to 98.7 wt. %, preferably in an amount in the range of 37 to 96.849 wt. %, particular preferred in an amount in the range of 48 to 93.79 wt. % and most preferred in an amount in the range of 61.5 to 90.55 wt. %, in each case based on the total weight of the composition (CR).

In a preferred embodiment component (F) comprises at least 50% by weight, more preferred at least 60% by weight, and particularly preferred at least 65% by weight of water, based on the total weight of component (F) comprised in the composition (CR).

### Component (G)

In the present case the terms “(G)”, “component (G)” and “at least one surfactant” are used synonymously, and therefore have preferably the same meaning. The term “at least one surfactant” means exactly one surfactant and, also a mixture of two or more different surfactants. Preferably as component (G) a mixture of two or more different surfactants is used.

The surfactants suitable as component (G) include anionic, cationic, amphoteric and non-ionic surfactants.

The anionic surfactants may be selected from the group consisting of salts (such as alkaline salts, for example, sodium salts, ammonium salts, amine salts, amino alcohol salts and magnesium salts) of the following compounds: fatty acids, alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, alpha-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; N-acyltaurates; and mixtures thereof. The alkyl or acyl radical of all of these various compounds, for example, comprises from 8 to 24 carbon atoms, and the aryl radical, for example, is chosen from phenyl and benzyl groups. Weakly anionic surfactants can also be used, such as alkyl-D-galactosiduronic acids and their salts, as well as polyoxyalkylenated (C6-C24)alkyl ether carboxylic acids, polyoxyalkylenated (C6-C24) alkylaryl ether carboxylic acids, polyoxyalkylenated (C6-C24) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

Suitable anionic surfactant(s) (Component (G)) may comprise at least one anionic functional group at their head selected from carboxylate, sulfate, sulfonate, and phosphate and carboxylates.

Suitable alkyl sulfates include ammonium lauryl sulfate, sodium lauryl sulfate (sodium dodecyl sulfate, SLS, or SDS), and alkyl-ether sulfates, such as sodium laureth sulfate (sodium lauryl ether sulfate or SLES), and sodium myreth sulfate.

Further suitable anionic surfactants may include docusate (dioctyl sodium sulfosuccinate), alkyl-aryl ether phosphate, alkyl ether phosphate, sodium lauroyl sarcosinate, ammonium laureth sulfate, disodium lauryl sulfosuccinate, and sodium lauryl sulphoacetate.

Preferred anionic surfactants may be selected from the group consisting of sodium laurylethersulfate, sodium laurethethersulfate, sodium dodecyl sulfate, ammonium laurethethersulfat, ammonium dodecyl sulfate, alkylbenzenesulfonate, and combinations thereof.

In a preferred embodiment component (G) is at least one cationic surfactant selected form the group consisting of Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, and Polyquaternium-47, and further quaternary ammonium compounds.

In a more preferred embodiment component (G) is at least one cationic surfactant selected form the group consisting of Polyquarternium-6, and Polyquarternium-11.

Suitable further quaternary ammonium compounds are preferably those selected from cetrimonium chloride, behentrimonium chloride, dipalmitoylethylhydroxyethylmonium methosulfate, behentrimonium methosulfate, and mixtures thereof.

The surfactant (component (G)) may be a non-ionic surfactant. The non-ionic surfactant may be selected from the group consisting of lanolin alcohol, and polyoxyethylene ethers of fatty alcohols, and mixtures thereof. The non-ionic surfactant may be preferably ceteareth-n, wherein n is from 2 to 100, or from 10 to 30. Suitable nonionic surfactants are compounds that are well known (see, for example, in this respect “Handbook of Surfactants” by M. R. Porter, published by Blackie & Son (Glasgow and London), 1991, pp. 116-178). Preferred surfactants encompass non-ionic surfactants, in particular polyoxyethylated fatty alcohols or oils. Particularly preferred non-ionic surfactants include polyoxyethylated Castor Oil and hydrogenated polyoxylated Castor Oil. Specific examples of non-ionic surfactants that may be used in the compositions of the present disclosure are PEG-35 Castor Oil, PEG-40 Hydrogenated Castor Oil, and combinations thereof.

In a more preferred embodiment component (G) is at least one surfactant selected form the group consisting of steareth-20, cetearyl alcohol, Polyquarternium-6, and Polyquarternium-11.

Component (G) is preferably present in an amount of from 0 to 15% by weight, more preferably in an amount of from 1 to 12% by weight, and particularly preferred in an amount of from 2 to 11% by weight, and most preferred in an amount of from 2.5 to 10, wherein the % by weight values are based on the total weight of the composition (CR).

### Component (H)

In the present case the terms “(H)”, “component (H)” and “at least one additional component” are used synonymously, and therefore have preferably the same meaning. The term “at least one additional component” means exactly one additional component and, also a mixture of two or more different additional component. Preferably as component (H) a mixture of two, three or more different additional components is used.

Composition (CR) may optionally comprise additional components which enhance, supplement, or add to the benefit obtained by components (C) and (D).

Such additional optional components will be referred to in the following as supportive components, and cosmetically acceptable additives.

According to embodiments, composition (CR) according to the present disclosure further may comprise a supportive component. Examples of such supportive components include in particular vasodilatators, blood circulation stimulators, antioxidants, vitamins, provitamins. Component (H) may be present in an amount 0 to 5 of percent-by-weight, based on the weight of composition (CR). If present, the amount of the supportive component or combinations thereof will be in the range of from 0.0010 to 5.0, in particular from 0.010 to 3.0 percent-by-weight, and most preferred in from 0.1 to 3 percent-by-weight, based on the weight of composition (CR).

The supportive component may comprise, for example, one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol (provitamin B5), omega-3 fatty acids, omega-6 fatty acids, folates, cannabidiol, amino acids, or combinations thereof.

According to embodiments, the supportive component includes a vasodilator selected from tocopherols (alpha-, beta-, gamma- or delta-tocopherol), in particular alpha-tocopherol, tocopherol esters (including, for example, tocopherol acetate, tocopheryl succinate, tocopherylnicotinate, tocopherylpoly(oxyethylene)-succinate), nicotinic acid, nicotinic amides, nicotinic esters, camphor; an antioxidant such as vitamin C or polyphenol(s); an inhibitor of testosterone-5-alpha-reductase such as the omega-6 fatty acid arachidonic acid; a substance providing a benefit to hair health such as nicotinic amide (niacinamide); or a combination thereof. According to embodiments, the supportive component includes tocopherol or tocopheryl acetate (vitamin E acetate), and/or niacinamide. Suitable vasodilatory plant extracts include burned or distilled nettle extract (Urtica Diocia), roast chestnut extract (Aesculus Hippocastanum), Arnica extract (Arnica Montana), or hemp extract.

Composition (CR) optionally may comprise cosmetically acceptable additives, which may contribute to achieve or enhance the properties or effects sought.

According to embodiments, the cosmetically acceptable additives may be selected from perfumes/fragrances, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents. The cosmetically acceptable additives may be used in any combinations, as desired or required.

Suitable cosmetically acceptable additives not specifically described below are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions), which are useful in identifying specific additives to achieve a particular purpose or multipurpose. A few of these additives are discussed below, whose disclosure is of course non-exhaustive.

Suitable amount ranges for perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents are indicated for additives discussed below. These cosmetically acceptable additives may be present in a total amount of up to 20.0, in particular up to 15.0 percent-by-weight, based on the weight of composition (CR). Typically, the total amount of these cosmetically acceptable additives may be within a range of from 0.010 to 10.0, conveniently within a range of from 0.010 to 7.0 percent-by-weight, based on the weight of composition (CR). For example, the total amount of these cosmetically acceptable additives may be within a range of from 0.010 to 5.0, conveniently within a range of from 0.010 to 3.0 percent-by-weight, based on the weight of composition (CR).

Composition (CR) used according to the present disclosure may comprise a perfume/fragrance. For example, composition (CR) may comprise from 0.0010 to 1.50, in particular 0.010-0.50 percent-by-weight perfume/fragrance, based on the weight of composition (CR). Perfume/fragrance can provide an enhanced user experience by making composition (CR) smell pleasant and/or invoke emotions, such as relaxing or exciting smells.

Alternatively, composition (CR) used according to the present disclosure may be substantially free of perfume and/or fragrance. Some consumers prefer perfume-free compositions. The perfume/fragrance may be an animal fragrance or a plant fragrance. The animal fragrance may be selected from the group consisting of musk oil, civet, castoreum, ambergris, and mixtures thereof.

The plant fragrance may be selected from the group consisting of nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

The perfume may comprise one or more scents selected from the group consisting of acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronnellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-α-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, and mixtures thereof.

Composition (CR) according to the present disclosure may comprise an opacifier, which sometimes is denoted as well by the term “turbidity-inducing agent”. For example, compositions may comprise from 0.10 to 5.0, in particular 0.50-2.0 percent-by-weight opacifier, based on the weight of composition (CR). Suitable opacifiers include mineral pigments such as, for example, titanium dioxide, alumina, calcium carbonate. Other suitable opacifiers include fatty alcohols and fatty acid esters such as, for example, cetearyl alcohol, cetyl alcohol, ethylene glycol distearate, glyceryl stearate, glyceryl oleate, stearyl stearate.

Composition (CR) according to with the present disclosure may comprise a preservative or mixture of preservatives. Compositions (CR) may comprise the preservative(s) in an amount of up to 1.0, for example from 0.0010 to 1.0, in particular from 0.010 to 0.50 percent-by-weight, based on the weight of composition (CR). Cosmetically acceptable preservatives include organic acids such as para-hydroxybenzoic acid; organic acid salts such as sodium benzoate; compounds such as benzyl alcohol, phenoxyethanol, 1,3-bis(hydroxymethyl)-5,5-dimethylimidazolidine-2,4-dione; capryl glycol, and mixtures thereof.

Composition (CR) according to the present disclosure may comprise a pH modifier and/or buffering agent, in a sufficient amount to effectively adjust the pH of the composition. For example, the compositions may comprise up to 1.0, for example from 0.000010 to 1.0, in particular 0.0010-0.50 percent-by-weight pH modifiers, based on the weight of the composition (CR). Suitable pH modifiers and/or buffering agents for use herein include, but are not limited to; and guanidium salts, alkali metal and ammonium hydroxides and carbonates, such as sodium hydroxide, sodium silicate, sodium meta silicate and ammonium carbonate, ammonium bicarbonate, ammonium chloride, and acids such as organic and inorganic acids, for example ascorbic acid, citric acid, acetic acid or tartaric acid, phosphoric acid, hydrochloric acid, and mixtures thereof.

Composition (CR) according to the present disclosure may further comprise one or more chelators (also known as “chelating agent”, “sequestering agent”, “sequestrant”, or chelants) in an amount sufficient to reduce the amount of metals available to interact with formulation components. Chelators are well known in the art and a non-exhaustive list thereof can be found in AE Martell & RM Smith, Critical Stability Constants, Vol. 1, Plenum Press, New York & London (1974) and AE Martell & RD Hancock, Metal Complexes in Aqueous Solution, Plenum Press, New York & London (1996). Typically, the compositions may comprise one or more chelators in a total amount of from 0.0010 to 1.0, in particular from 0.010 to 0.50 percent-by-weight, based on the weight of the composition (CR).

The one or more chelators may be selected from the group consisting of carboxylic acids (such as aminocarboxylic acids), phosphonic acids (such as aminophosphonic acids), polyphosphoric acids (such as linear polyphosphoric acids), their salts thereof, and mixtures thereof. By “salts thereof”, it is meant—in the context of chelators—all salts comprising the same functional structure as the chelators they are referring to and including alkali metal salts, alkaline earth salts, ammonium salts, substituted ammonium salts, and mixtures thereof; alternatively sodium salts, potassium salts, calcium salts, magnesium salts, ammonium salts, and mixtures thereof; alternatively monoethanolammonium salts, diethanolammonium salts, triethanolammonium salts, and mixtures thereof.

The one or more chelators may be one or more aminocarboxylic acid chelators comprising one or more carboxylic acid moieties (—COOH) and one or more nitrogen atoms. Suitable chelators may be selected from the group consisting of diethylenetriamine pentaacetic acid (DTPA), ethylenediamine disuccinic acid (EDDS), ethylenediamine-N,N′-diglutaric acid (EDDG), 2-hydroxypropylenediamine-N—N′-disuccinic acid (HPDDS), glycinamide-N,N′-disuccinic acid (GADS), ethylenediamine-N—N′-diglutaric acid (EDDG), 2-hydroxypropylenediamine-N—N′-disuccinic acid (HPDDS), ethylenediaminetetraacetic acid (EDTA), ethylenedicysteic acid (EDC), ethylenediamine-N—N′-bis(ortho-hydroxyphenyl acetic acid) (EDDHA), diaminoalkyldi(sulfosuccinic acids) (DDS), N,N′-bis(2-hydroxybenzyl)ethylenediamine-N,N′-diacetic acid (HBED), etidronic acid, diethylenetriamine penta(methylene phosphonic acid), and/or their salts thereof, and mixtures thereof.

Conveniently, a chelator used in the composition (CR) according to the present disclosure, if any, may be EDTA, etidronic acid, diethylenetriamine penta(methylene phosphonic acid) (DTPMP), and/or their salts thereof, and mixtures thereof.

Composition (CR) according to the present disclosure may comprise viscosity modifiers, referred to in the art as well as “thickening agents”. If present, such viscosity modifiers typically will be used in the composition (CR) in an amount of from 0.010 to 3.0, in particular from 0.50 to 1.0 percent-by-weight, based on the weight of the composition (CR). Suitable cosmetically acceptable viscosity modifiers include non-ionic polymers, for example polysaccharides such as cellulose, cellulose gum or xanthan gum, and non-ionic viscosity modifiers based on polyether-1 such as the commercially available viscosity modifiers Pure Thix 1442.

Composition (CR) according to the present disclosure may further comprise care materials, such as hair-care providing or skin-care-providing plant or vegetable extracts. Typical amounts of care materials in the compositions according to the present disclosure will be in the range of from 0.010 to 5.0, in particular 0.050-2.0 percent-by-weight, based on the weight of the composition (CR).

### Composition (CR) for Permanently Re-Forming Keratin Fibers

The composition (CR) for permanently re-forming keratin fibers comprising as components
(A) at least one thiol based reducing agent,
(B) at least one alkalizing agent,
(C) malic acid, and/or salts of malic acid,
(D) at least on betaine, and
(E) optionally at least one disulfide based compound.

In a preferred embodiment composition (CR) comprises as components
(A) at least one thiol based reducing agent, comprising ammonium thioglycolate and/or cysteine,
(B) at least one alkalizing agent, comprising ammonia and/or monoethanol amine,
(C) malic acid, and/or salts of malic acid,
(D) at least one betaine, comprising trimethylglycine, and
(E) optionally at least one disulfide based compound, comprising diammonium dithiodiglycolate.

In another preferred embodiment composition (CR) comprises as components
(A) at least one thiol based reducing agent, comprising at least 80% by weight of at least one thiol based reducing agent selected from the group consisting of ammonium thioglycolate and cysteine, based on the total weight of component (A) comprised in the composition (CR),
(B) at least one alkalizing agent, comprising at least 80% by weight of at least one alkalizing agent selected from the group consisting of ammonia and monoethanolamine based on the total weight of component (B) comprised in the composition (CR),
(C) malic acid, and/or salts of malic acid,
(D) at least one betaine, comprising 80% by weight of trimethylgycine, based on the total weight of component (D) comprised in the composition (CR), and
(E) optionally at least one disulfide based compound, comprising at least 80% by weight of diammonium dithiodiglycolate based, on the total weight of component (D) comprised in the composition (CR).

In more preferred embodiment composition (CR) comprises as components
(A) at least one thiol based reducing agent, comprising at least 90% by weight of at least one thiol based reducing agent selected from the group consisting of ammonium thioglycolate and cysteine, based on the total weight of component (A) comprised in the composition (CR),
(B) at least one alkalizing agent, comprising at least 90% by weight of at least one alkalizing agent selected from the group consisting of ammonia and monoethanolamine based on the total weight of component (B) comprised in the composition (CR),
(C) malic acid, and/or salts of malic acid,
(D) at least one betaine, comprising 90% by weight of trimethylglycine, based on the total weight of component (D) comprised in the composition (CR), and
(E) optionally at least one disulfide based compound, comprising at least 90% by weight of diammonium dithiodiglycolate based, on the total weight of component (D) comprised in the composition (CR).

In another more preferred embodiment composition (CR) comprises as components
(A) at least one thiol based reducing agent, comprising at least 95% by weight of at least one thiol based reducing agent selected from the group consisting of ammonium thioglycolate and cysteine, based on the total weight of component (A) comprised in the composition (CR),
(B) at least one alkalizing agent, comprising at least 95% by weight of at least one alkalizing agent selected from the group consisting of ammonia and monoethanolamine based on the total weight of component (B) comprised in the composition (CR),
(C) malic acid, and/or salts of malic acid,
(D) at least one betaine, comprising 95% by weight of trimethylglycine, based on the total weight of component (D) comprised in the composition (CR), and
(E) optionally at least one disulfide based compound, comprising at least 95% by weight of diammonium dithiodiglycolate based, on the total weight of component (D) comprised in the composition (CR).

In a particularly preferred embodiment, the composition (CR) comprises as components
(A) ammonium thioglycolate and/or cysteine,
(B) ammonia and/or monethanolamine,
(C) malic acid, and/or salts of malic acid,
(D) trimethylgycine, and
(E) optionally diammonium dithiodiglycolate.

In one embodiment the composition (CR) comprises
component (A) in an amount of from 1 to 15% by weight, preferably in an amount from 1.5 to 14, particularly preferred in an amount from 3 to 13% by weight, and most preferred in an amount from 5 to 13.5% by weight,
component (B) in an amount of from 0.1 to 5% by weight, preferably in an amount from 0.15 to 4, particularly preferred in an amount from 0.2 to 3% by weight, and most preferred in an amount from 0.25 to 2% by weight,
component (C) in an amount of from 0.1 to 15% by weight, preferably in an amount from 0.2 to 10, particularly preferred in an amount from 0.25 to 8% by weight, and most preferred in an amount from 0.3 to 3% by weight,
component (D) in an amount of from 0.1 to 15% by weight, preferably in an amount from 0.2 to 10, particularly preferred in an amount from 0.25 to 8% by weight, and most preferred in an amount from 0.3 to 3% by weight,
component (F) in an amount of from 30 to 98.7% by weight, preferably in an amount from 45 to 96.949% by weight, particularly preferred in an amount from 54 to 94.29% by weight, and most preferred in an amount from 65.5 to 91.55% by weight,
component (G) in an amount of from 0 to 15% by weight, preferably in an amount from 1 to 12% by weight, particularly preferred in an amount from 2 to 11% by weight, and most preferred in an amount from 2.5 to 10% by weight, and
component (H) in an amount of from 0 to 5% by weight, preferably in an amount from 0.001 to 5% by weight, particularly preferred in an amount from 0.01 to 3% by weight, and most preferred in an amount from 0.1 to 3% by weight,
wherein the % by weight values are based on the total weight of the composition (CR).

In another embodiment the composition (CR) comprises
component (A) in an amount of from 1 to 15% by weight, preferably in an amount from 1.5 to 14, particularly preferred in an amount from 3 to 13% by weight, and most preferred in an amount from 5 to 13.5% by weight,
component (B) in an amount of from 0.1 to 5% by weight, preferably in an amount from 0.15 to 4, particularly preferred in an amount from 0.2 to 3% by weight, and most preferred in an amount from 0.25 to 2% by weight,
component (C) in an amount of from 0.1 to 15% by weight, preferably in an amount from 0.2 to 10, particularly preferred in an amount from 0.25 to 8% by weight, and most preferred in an amount from 0.3 to 3% by weight,
component (D) in an amount of from 0.1 to 15% by weight, preferably in an amount from 0.2 to 10, particularly preferred in an amount from 0.25 to 8% by weight, and most preferred in an amount from 0.3 to 3% by weight,
component (E) in an amount of from 0 to 10% by weight, preferably in an amount from 0.1 to 8, particularly preferred in an amount from 0.5 to 6% by weight, and most preferred in an amount from 1 to 4% by weight,
component (F) in an amount of from 20 to 98.7% by weight, preferably in an amount from 37 to 96.849% by weight, particularly preferred in an amount from 48 to 93.79% by weight, and most preferred in an amount from 61.5 to 90.55% by weight,
component (G) in an amount of from 0 to 15% by weight, preferably in an amount from 1 to 12% by weight, particularly preferred in an amount from 2 to 11% by weight, and most preferred in an amount from 2.5 to 10% by weight, and
component (H) in an amount of from 0 to 5% by weight, preferably in an amount from 0.001 to 5% by weight, particularly preferred in an amount from 0.01 to 3% by weight, and most preferred in an amount from 0.1 to 3% by weight,
wherein the % by weight values are based on the total weight of the composition (CR).

The composition (CR) for permanently re-forming keratin fibers can be used for permanently waving keratin fibers or for permanently straightening keratin fibers.

Another aspect of the present invention, therefore, is the use of the composition (CR) for permanently waving keratin fibers.

Another aspect of the present invention is the use of the composition (CR) for permanently straightening keratin fibers.

The pH value of the composition (CR) is preferably in the range of 7.5 to 10.0, more preferred in the range of 7.8 to 9.8, even more preferred in the range of 7.9 to 9.6.

Another aspect of the present invention is a kit for permanently re-forming keratin fibers, comprising the composition (CR) as described herein, and a neutralizing composition (CN) comprising at least one oxidizing agent.

For the composition (CR) comprised in the kit, the above description and preferences hold true accordingly.

The neutralizing composition (CN) comprises at least one oxidizing agent. The neutralizing composition is capable to crosslink the cleaved cysteine bonds again which were removed by the application of the composition (CR), namely the thiol based reducing agent (component (A)) contained therein.

Suitable neutralizing compositions and oxidizing agents are known to the person skilled in the art. Typically the neutralizing composition (CN) comprises hydrogen peroxide and or bromate compounds as an oxidizing agent.

Suitable bromate compounds are for example selected form ammonium bromate, and/or alkali bromates, like sodium bromate.

Another aspect of the present invention is a process for permanently re-forming keratin fibers, comprising the steps
i) applying the composition (CR) for permanently re-forming keratin fibers as described herein to keratin fibers,
ii) re-forming the keratin fibers, and
iii) applying the neutralizing composition (CN) comprising at least one oxidizing agent to the keratin fibers.

Another aspect of the present invention is a process for permanently waving keratin fibers, preferably human hair, comprising the steps
w1) washing the keratin fibers, preferably the human hair,
w2) rolling the keratin fibers, preferably the human hair, in wet state onto a roller,
w3) applying the composition (CR) as described herein, onto the keratin fibers, preferably onto the human hair, in the roller for a time period in the range of 5 to 25 minutes,
w4) rinsing off the composition (CR) from the rolled keratin fibers, preferably from the rolled human hair,
w5) optionally padding dry the rolled keratin fibers, preferably the rolled human hair,
w6) applying the neutralizer composition (CN) as described herein onto the rolled keratin fibers in the roller, preferably onto the rolled human hair in the roller,
w7) removing the keratin fiber, preferably the human hair, from the roller,
w8) optionally applying another portion of the neutralizer composition (CN) onto the keratin fibers, preferably onto the human hair,
w9) rinsing the keratin fibers, preferably the human hair,
w10) optionally applying a conditioner onto the keratin fibers, preferably onto the human hair, and
w11) optionally blow drying the keratin fibers, preferably the human hair.

Step w3) can be conducted with or without heating. Preferably step w3) is conducted with heating in a climate zone, wherein temperatures in the range of 25° C. to 45° C. are applied to the keratin fibers, preferably to the human hair. It is also possible to use heated rollers (curlers) which are heated to temperatures in the range from 70 to 180° C., more preferred in the range of 70 to 170° C. and even more preferred in the range of 75 to 140° C.

Another aspect of the present invention is a process for permanently straightening keratin fibers, preferably human hair, comprising the steps
s1) applying the composition (CR) as described herein, onto the keratin fibers, preferably onto the human hair, in the roller for a time period in the range of 3 to 30 minutes,
s2) rinsing off the composition (CR) from the keratin fibers, preferably from the human hair,
s3) blow drying the keratin fibers, preferably from the human hair,
s4) straightening the keratin fibers, preferably the human hair, by
   i) using a flat iron on the keratin fibers, preferably on the human hair, or
   ii) combing keratin the fibers, preferably the human hair,
s5) applying the neutralizer composition (CN) as described herein onto the keratin fibers, preferably onto the human hair,
s6) rinsing the keratin fibers, preferably the human hair,
s7) optionally applying a conditioner onto the keratin fibers, preferably onto the human hair, and
s8) optionally blow drying the keratin fibers, preferably the human hair.

### EMBODIMENTS

1. Composition (CR) for permanently re-forming keratin fibers comprising as components
   (A) at least one thiol based reducing agent,
   (B) at least one alkalizing agent,
   (C) malic acid, and/or salts of malic acid,
   (D) at least one betaine, and
   (E) optionally at least one disulfide based compound.
2. Composition (CR) for permanently re-forming keratin fibers according to embodiment 1, wherein component (A) is at least one thiol based reducing agent selected from the group consisting of thioglycolic acid, ammonium thioglycolate, thiolactic acid, glycerol monothiolactate, ammonium thiolactate, MEA-thiolactate, 3-mercaptopropionic acid, glycerol 3-mercaptopropionate, ethyleneglycol 3-mercaptopropionate, cysteamine, N-acetylcysteamine, N-propionylcysteamine, cysteine, N-acetylcysteine, N-alkanoylcysteine, cysteine alkyl esters, homocysteine, thioglycolic acid, ethanolamine thioglycolate, glyceryl thioglycolate, glutathione, thioglycerol, thiomalic acid, thiodiglycol, 2-mercaptoethanol, dithiothreitol, thioxanthine, thiosalicylic acid, thiopropionic acid, lipoic acid, and mixtures thereof.
3. Composition (CR) for permanently re-forming keratin fibers according to embodiment 1 or 2, wherein component (A) is at least one thiol based reducing agent selected from the group consisting of thioglycolic acid, ammonium thioglycolate, cysteine, and mixtures thereof.
4. Composition (CR) for permanently re-forming keratin fibers according to any one of embodiments 1 to 3, wherein component (B) is at least one alkalizing agent selected from the group consisting of 2-amino-1-propanol, 2-amino-1-butanol, 2-dimethylamino-2-pethyl-1-propanol, 2-amino-2 -methylpropan-1-ol, 3-amino-1-propanol, ammonia, and monoethanolamine.
5. Composition (CR) for permanently re-forming keratin fibers according to any one of embodiments 1 to 4, wherein the composition additionally comprises as component (F) is at least one cosmetically acceptable solvent.
6. Composition (CR) for permanently re-forming keratin fibers according to any one of embodiments 1 to 5, wherein component (D) at least one betaine selected from the group consisting of alkylbetaines, alkylamidopropylbetaines and alkylsulfobetaines.
7. Composition (CR) for permanently re-forming keratin fibers according to any one of embodiments 1 to 6, wherein component (D) is at least one betaine of the formula (I)

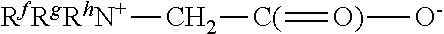

wherein R^{f}, R^{g} and R^{h} are each, same or different, selected from the group consisting of C1-C6 alkyl, C1-C6 hydroxyalkyl and C1-C6 carboxyalkyl.
8. Composition (CR) for permanently re-forming keratin fibers according to any one of embodiments 1 to 7 wherein component (D) is trimethylglycine.
9. Composition (CR) for permanently re-forming keratin fibers according to any one of embodiments 1 to 8, wherein the pH value of the composition (CR) is in the range of 7.5 to 10.0.
10. Composition (CR) for permanently re-forming keratin fibers according to any one of embodiments 1 to 9, wherein the composition (CR) comprises from 0.1 to 15% by weight of component (C), based on the total weight of the composition (CR).
11. Composition (CR) for permanently re-forming keratin fibers according to any one of embodiments 1 to 10, wherein the composition comprises 0.1 to 15% by weight of component (D), based on the total weight of the composition (CR).
12. Composition (CR) for permanently re-forming keratin fibers according to any one of embodiments 1 to 11, wherein the composition comprises 1 to 15% by weight of component (A), based on the total weight of the composition (CR).
13. Composition (CR) for permanently re-forming keratin fibers according to any one of embodiments 1 to 12, wherein the composition comprises 0.1 to 5% by weight of component (B), based on the total weight of the composition (CR).
14. A kit for permanently re-forming keratin fibers, comprising the composition (CR) for permanently re-forming keratin fibers according to any of embodiments 1 to 13 and a neutralizing composition (CN) comprising at least one oxidizing agent.
15. A process for permanently re-forming keratin fibers, comprising the steps
   i) applying the composition (CR) for permanently re-forming keratin fibers according to any of embodiments 1 to 13 to keratin fibers,
   ii) re-forming the keratin fibers, and
   iii) applying the neutralizing composition (CN) comprising at least one oxidizing agent to the keratin fibers.

### EXAMPLES

The following examples illustrate the composition (CR) and performing results of said composition. These examples are not intended to be inclusive of all aspects of the subject matter disclosed herein, but rather to illustrate representative compositions (CR) and results. These examples are not intended to exclude equivalents and variations of the presently claimed invention, which are apparent to one skilled in the art.

To evaluate the protective effect of malic acid and betaine in the inventive composition (CR) for permanently re-forming keratin fibers formulations that only differ in their malic acid and betaine content were applied to hair tresses.

Natural, bleached Caucasian hair was used (Supplier Kerling).

The compositions are shown in table 1 below. Composition CR1 is a composition according to the present invention. Compositions C2 to C4 are comparative examples. Composition C2 contains only malic acid. Composition C3 contains only betaine. Composition C4 contains neither malic acid nor betaine.

| | | CR1 | C2 | C3 | C4 |
|---|---|---|---|---|---|
| Ammonium | Component (A) | 12.68% | 12.68% | 12.68% | 12.68% |
| Thioglycolate | | | | | |
| Diammonium | Component (E) | 1.92% | 1.92% | 1.92% | 1.92% |
| Dithiodiglycolate | | | | | |
| Ammonia | Component (B) | 1.08% | 1.08% | 0.83% | 0.83% |
| Ammonium | Component (H) | 1.00% | 1.00% | 1.00% | 1.00% |
| Bicarbonate | | | | | |
| Polyquaternium-6 | Component (G) | 0.80% | 0.80% | 0.80% | 0.80% |
| Steareth-20 | Component (G) | 2.50% | 2.50% | 2.50% | 2.50% |
| Cetearyl Alcohol | Component (G) | 6.00% | 6.00% | 6.00% | 6.00% |
| Panthenol | Component (H) | 0.35% | 0.35% | 0.35% | 0.35% |
| Fragrance | Component (H) | 1.00% | 1.00% | 1.00% | 1.00% |
| Malic Acid | Component (C) | 1.00% | 1.00% | | |
| Betaine | Component (D) | 0.87% | | 0.87% | |
| Water | Component (F) | 70.81% | 71.67% | 72.06% | 72.93% |
| TOTALS: | | 100.00% | 100.00% | 100.00% | 100.00% |

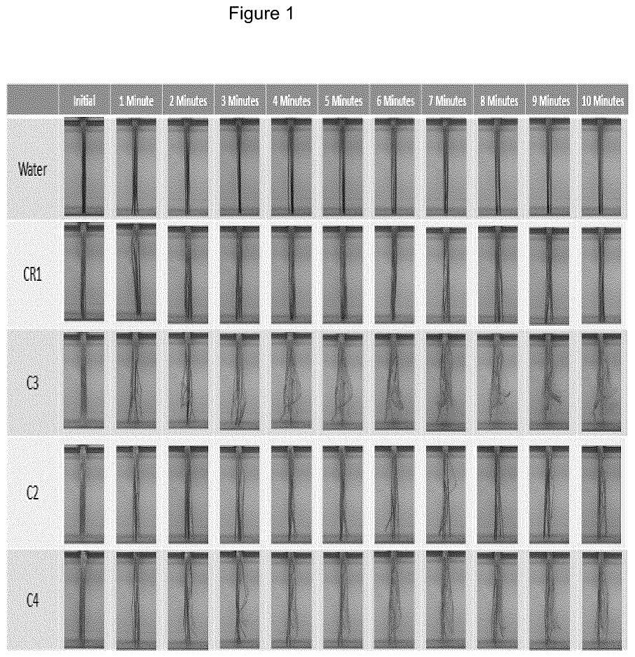

The degree of hair degradation was captured by taking pictures directly after application (Initial) and after 1, 2, 3, 4, 5, 6, 7, 8, 9, and 10 minutes. The pictures were compared among each other (FIG. 1).

FIG. 1 shows in the first row of the table the results for water which serves as reference. In rows two to four of table in FIG. 1 the results for the compositions CR1, and C2, C3, and C4 are depicted.

According to FIG. 1, the performance of the tested compositions can be ranked in the following order from best to worst:
1. The composition CR1 according to the invention shows the best results. There is almost no damage as the pictures of the tresses look similar to the tresses on which only water was applied.
2. The composition C2 (only malic acid) shows little damage, but composition C2 performs worse than the inventive composition CR1.
3. The composition C4 (no malic acid/no betaine) shows damage, but composition C4 performs worse than composition C2.
4. The composition C3 (only betaine) shows serious damage and performs significantly worse than composition C4.

Because composition C3 (only betaine) performs worse than composition C4 (no malic acid/no betaine), and at the same time composition CR1 according to the invention (malic acid and betaine) performs better than composition C2 (only malic acid), a synergistic effect of malic acid and betaine is shown, instead of only an additive effect.

1. Composition (CR) for permanently re-forming keratin fibers comprising as components
(A) at least one thiol based reducing agent,
(B) at least one alkalizing agent,
(C) malic acid, and/or salts of malic acid,
(D) at least one betaine, and
(E) optionally at least one disulfide based compound.

2. Composition (CR) for permanently re-forming keratin fibers according to claim 1, wherein component (A) is at least one thiol based reducing agent selected from the group consisting of thioglycolic acid, ammonium thioglycolate, thiolactic acid, glycerol monothiolactate, ammonium thiolactate, MEA-thiolactate, 3-mercaptopropionic acid, glycerol 3-mercaptopropionate, ethyleneglycol 3-mercaptopropionate, cysteamine, N-acetylcysteamine, N-propionylcysteamine, cysteine, N-acetylcysteine, N-alkanoylcysteine, cysteine alkyl esters, homocysteine, thioglycolic acid, ethanolamine thioglycolate, glyceryl thioglycolate, glutathione, thioglycerol, thiomalic acid, thiodiglycol, 2-mercaptoethanol, dithiothreitol, thioxanthine, thiosalicylic acid, thiopropionic acid, lipoic acid, and mixtures thereof.

3. Composition (CR) for permanently re-forming keratin fibers according to claim 1, wherein component (A) is at least one thiol based reducing agent selected from the group consisting of thioglycolic acid, ammonium thioglycolate, cysteine, and mixtures thereof.

4. Composition (CR) for permanently re-forming keratin fibers according to claim 1, wherein component (B) is at least one alkalizing agent selected from the group consisting of 2-amino-1-propanol, 2-amino-1-butanol, 2-dimethylamino-2-pethyl-1-propanol, 2-amino-2-methylpropan-1-ol, 3-amino-1-propanol, ammonia, and monoethanolamine.

5. Composition (CR) for permanently re-forming keratin fibers according to claim 1, wherein the composition additionally comprises as component (F) is at least one cosmetically acceptable solvent.

6. Composition (CR) for permanently re-forming keratin fibers according to claim 1, wherein component (D) at least one betaine selected from the group consisting of alkylbetaines, alkylamidopropylbetaines and alkylsulfobetaines.

7. Composition (CR) for permanently re-forming keratin fibers according to claim 1, wherein component (D) is at least one betaine of the formula (I) 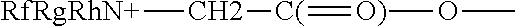
wherein Rf, Rg and Rh are each, same or different, selected from the group consisting of C1-C6 alkyl, C1-C6 hydroxyalkyl and C1-C6 carboxyalkyl.

8. Composition (CR) for permanently re-forming keratin fibers according to claim 1 wherein component (D) is trimethylglycine.

9. Composition (CR) for permanently re-forming keratin fibers according to claim 1, wherein the pH value of the composition (CR) is in the range of 7.5 to 10.0.

10. Composition (CR) for permanently re-forming keratin fibers according to claim 1, wherein the composition (CR) comprises from 0.1 to 15% by weight of component (C), based on the total weight of the composition (CR).

11. Composition (CR) for permanently re-forming keratin fibers according to claim 1, wherein the composition comprises 0.1 to 15% by weight of component (D), based on the total weight of the composition (CR).

12. Composition (CR) for permanently re-forming keratin fibers according to claim 1, wherein the composition comprises 1 to 15% by weight of component (A), based on the total weight of the composition (CR).

13. Composition (CR) for permanently re-forming keratin fibers according to claim 1, wherein the composition comprises 0.1 to 5% by weight of component (B), based on the total weight of the composition (CR).

14. A kit for permanently re-forming keratin fibers, comprising the composition (CR) for permanently re-forming keratin fibers according to claim 1 and a neutralizing composition (CN) comprising at least one oxidizing agent.

15. A process for permanently re-forming keratin fibers, comprising the steps
i) applying the composition (CR) for permanently re-forming keratin fibers according to claim 1 to keratin fibers,
ii) re-forming the keratin fibers, and
iii) applying the neutralizing composition (CN) comprising at least one oxidizing agent to the keratin fibers.
